(19) United States
(12) Patent Application Publication
(10) Pub. No.: US 2006/0093578 A1
(43) Pub. Date: May 4, 2006

(54) Method and composition for treating multiple sclerosis

(75) Inventor: Akio Suzumura, Nagoya-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kyorin Pharmaceutical Co., Ltd., Tokyo (JP)

(21) Appl. No.: 11/266,409

(22) Filed: Nov. 4, 2005

Related U.S. Application Data
(60) Provisional application No. 60/624,851, filed on Nov. 4, 2004.

(51) Int. Cl. A61K 38/21; A61K 31/519

(52) U.S. Cl. 424/85.6; 514/252.16

(57) ABSTRACT

A method of treating multiple sclerosis including administering Interferon-β and a phosphodiesterase inhibitor in combination in a therapeutically effective amount.

### CROSS-REFERENCE TO RELATED APPLICATIONS

The present application claims the benefit of U.S. Provisional Application No. 60/624,851, filed Nov. 4, 2004. The contents of that application are incorporated herein by reference in their entirety.

### BACKGROUND OF THE INVENTION

1. Field of the Invention

The present invention relates to a method and composition for treating multiple sclerosis and a method of modulating effects of Interferon-β on microglia.

2. Discussion of the Background

Multiple sclerosis (MS) is a disease in which neuronal axons are demyleinated within the central nervous system eventually leading to motor deficiencies and debilitation in patients. It is thought that there may be an auto-immune component to the disease such that the immune system attacks the oligodendrocytes that form the myelin sheaths that surround axons. As a major component of the neuroimmune system, microglia have been implicated in the etiology and expression of MS.

Although the pathogenesis of MS still remains to be elucidated, tumor necrosis factor α (TNFα) and/or free radicals (e.g., NO and superoxide) may play a critical role in development of inflammatory demyelination. MS is also considered to be mediated by type I helper T cells (Th1), which secrete interferon γ (IFNγ), interleukin-2 (IL-2), and TNFα. In order to differentiate to Th1, naive helper T cells require signals from antigen presenting cells. One of the most critical signals for this differentiation is IL-12. Therefore, suppression of IL-12 production by antigen-presenting cells may interfere with differentiation of Th1, resulting in suppression of Th1-mediated autoimmune diseases.

Interferon-β (IFNβ) is used to treat the relapsing-remitting form of MS. However, the exact mechanisms on how IFNβ exerts its function to reduce exacerbation of MS still remains to be elucidated. It has been reported to down-regulate IFNγ-induced class II MHC antigen expression on antigen presenting cells (APC), such as dendritic cells, macrophages, glial cells or endothelial cells (Inaba K, Kitaura M, Kato T, et al. “Contrasting effects of α/β and γ-interferon on expression of macrophage Ia antigens.” J Exp Med 1986;163:1030-1035.; Joseph J, Knobler R L D'Imperio C, Lublin F D. “Down regulation of interferon-γ induced class II expression on human glioma cell by recombinant interferon-β: effects of dosage treatment schedule.” J Neuroimmunol 1988;20:39-44.; Jiang I F, Milo R, Swoveland P, et al. “Interferon b-1b reduces interferon-γ-induced antigen presenting capacity of human glial and B cells.” J Neuroimmunol 1995;61:17-25, the contents of which are hereby incorporated by reference in their entirety).

It has also been shown that IFNβ suppresses IL-12 production with dendritic cells (van Seventer J M, Nagai T, van Seventer G A. “Interferon-β differentially regulates expression of the IL 12 family members p35, p40, p19 and EB13 in activated human dendritic cells.” J Neuroimmunol 2002;133:60-71, the contents of which are hereby incorporated by reference in their entirety), though the effects on IL-12 production by microglia remain to be clarified. Since IL-12 is a critical cytokine to induce development of T helper 1 (Th1), suppression of IL-12 production by APC may interfere with differentiation of Th1, resulting in suppression of Th1-mediated autoimmune diseases, such as MS. IFNβ is also an antiproliferative agent to suppress proliferation of T cells (Killestein J, Hintzen R Q, Uitdehaag B M, et al. “Baseline T cell reactivity in multiple sclerosis is correlated to efficacy of interferon-beta.” J Neuroimmunol 2002; 133:217-24).

Thus, IFNβ may inhibit both the processes of antigen presentation and clonal expansion of pathogenic T cells. However, whether or not IFNβ actually reduces the Th1 response is quite controversial. Some studies have shown decrease of Th1 cytokines or increase of Th2 cytokines in IFNβ-treated MS patients (Rudick R A, Ransofoff R M, Peppler R, et al. “Interferon beta induces interleukin-10 expression: relevance to multiple sclerosis.” Ann Neurol 1996;40:618-627; Yong V W, Chabot S, Stuve O, Williams G. “Interferon beta in the treatment of multiple sclerosis: mechanisms of action.” Neurology 1998;51:682-689). However, those results have not been confirmed by others (Dayal A S, Jensen B S, Liedo A, Arnason B G W “Interferon-gamma-secreting cells in multiple sclerosis patients treated with interferon beta-1b.” Neurology 1995;45:2173-2177). Furthermore, it has been shown recently that type 1 interferons (α/β) are major factors leading to Th1 development (Farrar J D, Murphy K M. “Type I interferons and T helper development.” Immunol Today 2000;21 :486-489).

TNFα and/or nitric oxide (NO) may play a critical role in development of inflammatory demyelination (Selmaj K W, Raine C S, Farooq M. “Cytokine cytotoxicity against oligodendrocytes. Apoptosis induced by lymphotoxin.” J Immunol 1991;147: 1522-1529; Selmaj K, Raine C S, Cannella B, Brosnan C F. “Identification of lymphotoxin and tumor necrosis factor in multiple sclerosis lesions.” J Clin Invest 1991;87:949-954; Merrill J., Ignarro L J, Sherman M P, Melinek J, Lane T E. “Microglial cell cytotoxicity of oligodendrocytes is mediated through nitric oxide.” J Immunol 1993; 151: 2132-2141). As has been shown, microglia are main producers of TNFα, superoxide, and NO in the CNS (Sawada M, Kondo N, Suzumura A, Marunouchi T. “Production of tumor necrosis factor-alpha by microglia and astrocytes in culture.” Brain Res 1989;491:394-397; Suzumura A, Sawada M. “Microglia as immunoregulatory cells in the central nervous system.” E A Eng, et al (eds) Topical Issues in Microglia Research, Singapore Neuroscience Association, Singapore, 1996; 189-202). Suppression of microglia-derived TNFα reportedly suppressed inflammatory demyelination (Selmaj K W, Raine C S. “Experimental autoimmune encephalomyelitis: immunotherapy with anti-tumor necrosis factor antibodies and soluble tumor necrosis factor receptors.” Neurology 1995;45(Suppl 6):S44-49; Klinkert W E, Kojima K, Lesslauer W, et al. “TNF-alpha receptor fusion protein prevents experimental auto-immune encephalomyelitis and demyelination in Lewis rats: an overview.” J Neuroimmunol 1997;72:163-8), suggesting the critical functions of microglia on development of MS pathology. However, TNFα neutralization with antibodies or binding of cytokine with a recombinant TNF receptor p55 immunoglobulin fusion protein led to increase in relapse in MS patients (van Oosten B W, Barkhof F, Frayen L, et al. “Increased MRI activity and immune activation in two multiple sclerosis patients treated with the monoclonal anti-tumor necrosis factor antibody cA2.” Neurology 1996;47:1531-1534; The Lanercept Multiple Sclerosis Study Group and the University of British Columbia MS/MRI Analysis Group. “TNF neutralization in MS: results of a placebo-controlled multicenter study.” Neurology 1999;53:457-465). Thus, the effects of TNFα on development of MS lesions also await further elucidation.

Effects of IFNβ on production of pro-inflammatory mediators, such as TNFα, IL-1, IL-6 and NO are also controversial (Abu-Khabar K S, Armstrong J A, Ho M. “Type I interferons (IFN-α and -β) suppress cytotoxin (tumor necrosis factor-α and lymphotoxin) production by mitogen-stimulated human peripheral blood mononuclear cells.” J Leukocyte Biol 1992;52:165-172; Chabot S, Williams G, Yong V W. “Microglial production of TNF-α, is induced by activated T lymphocytes; involvement of VLA-4 and inhibition by interferonβ-b.” J Clin Invest 1997; 100:604-612; Guathikonda P, Baker J, Mattson D H. “Interferon-beta-1-b (IFN-β) decreases induced nitric oxide (NO) production by a human astrocytoma cell line.” J Neuroimmunol 1998;82:133-139). Although IFNβ-treatment reportedly reduced mRNA expression for TNFα in peripheral blood mononuclear cells (PBMC; Ossege L M, Sindern E, Voss B, Malin J P “Immunomodulatory effects of IFNβ-1b on the mRNA-expression of TGFβ-1 and TNFα in vitro.” Immunopharmacol 1999;43:39-46), higher expression of TNFα mRNA in PBMC of IFNβ-treated MS patients has also been shown (Sarchielli P, Critelli A, Greco L, et al. “Expression of TNF-alpha mRNA by peripheral blood mononuclear cells of multiple sclerosis patients treated with IFN-beta IA.” Cytokine 2001;14:294-298).

Furthermore, effects of IFNβ in the central nervous system (CNS) await further elucidation. As noted, microglia can function as APC in the CNS. Although they usually do not express class II MHC antigen on their surface, cytokines from activated T cells, such as INFγ have been shown to induce class II MMC antigens on their surface. IFNγ also up-regulates the expression of co-stimulatory molecules, such as B7-1 or B7-2 required for complete activation of T cells (Satoh J, Lee Y B, Kim S U. “T-cell costimulatory molecules B71 (CD80) and B7-2 (CD86) are expressed in human microglia but not in astrocytes in culture.” Brain Res 1995; 704: 92-96; Menendez Iglesias B, Cerase J, Ceracchini C, Levi G, Aloisi F. “Analysis of B7-1 and B7-2 costimulatory ligands in cultured mouse microglia: upregulation by interferon-gamma and lipopolysaccharide and downregulation by interleukin-10, prostaglandin E2 and cyclic AMP-elevating agents.” J Neuroimmunol 1997;72: 83-93).

It has been shown that phosphodiesterase inhibitors (PDEIs) effectively suppress the production of inflammatory mediators such as TNFα, nitric oxide, and superoxide by glial cells (A. Suzumura, M. Sawada, M. Makino, T. Takayanagi, “Propentofylline inhibits production of TNFoc and infection of LP-BM5 murine leukemia virus in glial cells.” J Neurovirol, 4 (1998) 553-559; M. Yoshikawa, M., A. Suzumura T. Tamaru, T. Takayanagi, M. Sawada, “Effects of phosphodiesterase inhibitors on microglia.” Multiple Sclerosis, 5 (1999) 126-133; A. Suzumura, A. Ito, M. Yoshikawa, M. Sawada, “lbudilast suppresses TNFoc production by glial cells functioning mainly as type III phosphodiesterase inhibitor in the CNS.” Brain Res., 837 (1999) 203-21; A. Suzumura and M. Sawada, “Effects of vesnarinone on cytokine production and activation of murine microglia.” Life Sci., 64 (1999) 1197-1203; M. Yoshikawa, A. Suzumura, A. Ito, T. Tamaru, T. Takayanagi, “Effects of phosphodiesterase inhibitors on nitric oxide production by glial cells.” Tohoku J. Exp. Med. 196 (2002) 167-177), macrophages, and lymphocytes (C. S. Kasyapa, C. L. Stentz, M. P. Davey, D. W Carr, “Regulation of IL-15-stimulated TNF-alpha production by rolipram.” J Immunol. 163(1999) 2836-43; J. L. Jimenez, C. Punzon, J. Navarro, M. A. Munoz-fernandez, M. Fresno, “Phosphodiesterase 4 inhibitors prevent cytokine secretion by T lymphocytes by inhibiting nuclear factor-kappaB and nuclear factor of activated T cells activation.” J Pharmacol. Exp. Ther., 299(2001) 753-9). Since other cAMP-elevating agents or dibutyryl cAMP have the same effects on these cells (Yoshikawa et al., supra), PDEIs are considered to exert the above suppressive effects through elevation of intracellular cAMP, which results in suppression of translocation of nuclear factor NF-KB into nuclei (G. C. N. Perry and N. Mackman, “Role of cAMP response element-binding protein in cAMP inhibition of NF-kB-mediated transcription.” J. Immunol., 159 (1997) 5450-5456). Some PDEIs have been shown to suppress development of experimental allergic encephalomyelitis (EAE), which is an animal model of MS (O. Rott, E. Cash, B. Fleischer, “Phosphodiesterase inhibitor pentoxifylline, a selective suppressor of T helper type 1- but not type 2-associated lymphokine production, prevents induction of experimental autoimmune encephalomyelitis in Lewis rats.” Eur. J. Immunol., 23(1993) 1745-51; T. Fujimoto, S. Sakoda, H. Fujimura, T. Yanagihara, “Ibudilast, a phosphodiesterase inhibitor, ameliorates experimental autoimmune encephalomyelitis in Dark August rats.” J. Neuroimmunol., 95(1999) 35-42; H. Dinter, J. Tse, M. Halks-Miller, D. Asarnow, J. Onuffer, D. Faulds, B. Mitrovic, G. Kirsch, H. Laurent, P. Esperling, D. Seidelmann, E. Ottow, H. Schneider, V K. Tuohy, H. Wachtel, H. D. Perez, “The type IV phosphodiesterase specific inhibitor mesopram inhibits experimental autoimmune encephalomyelitis in rodents.” J. Neuroimmunol., 108(2000) 136-46). In a pilot study, PDEI also effectively suppressed the relapse rate of MS, when used in a combination of 3 different types (A. Suzumura, T. Nakamuro, T. Tamaru, T. Takayanagi, “Drop in relapse rate of multiple sclerosis patients using combination therapy of three different phosphodiesterase inhibitors.” Multiple Sclerosis, 6 (2000) 56-58, the contents of which are hereby incorporated by reference in their entirety).

Analysis of cytokine profile in peripheral blood CD4+T cells during the treatment revealed that the expression of Th1 type cytokines such as IFNγ and IL-2 decreased while Th2 type cytokines such as IL-4 and IL-10 increased, indicating that PDEIs induced immune deviation into Th2 type responses [Kikui et al. submitted for publication]. Similar immune deviation from a Th1 to Th2 dominant state was observed in patients with HTLV-1 associated myelopathy treated with PDEI pentoxifylline (T. Fujimoto, T. Nakamura, T. Furuya, S. Nakane, S. Shirabe, C. Kambara, S. Hamasaki, T. Yoshimura, K. Eguchi, “Relationship between the clinical efficacy of pentoxifylline treatment and elevation of serum T helper type 2 cytokine levels in patients with human T-lymphotropic virus type I-associated myelopathy.” Intern. Med., 3 8 (1999) 717-21), and in the patients with MS during IFNβ treatment (R. A. Rudick, R. M. Ransohoff, J. C. Lee, R. Peppler, M. Yu, P. M. Mathisen, V K. Touhy, “In vivo effects of interferon beta-1a on immunosuppressive cytokines in multiple sclerosis.” Neurology 50 (1998) 1294-1300). However, the mechanisms of how PDEIs or IFNβ induce immune deviation are still unclear. In order to provide compositions and methods for the treatment of MS, the effects of PDEIs and other cAMP-elevating agents, as well as IFNβ on IL-12 production with microglia are examined below.

### SUMMARY OF THE INVENTION

According to one aspect of the present invention, a method of treating multiple sclerosis includes administering Interferon-β and one or more phosphodiesterase inhibitors in combination in a therapeutically effective amount.

According to another aspect of the present invention, a composition for treating multiple sclerosis includes Interferon-β and one or more phosphodiesterase inhibitors. The Interferon-β and one or more phosphodiesterase inhibitors are provided in a therapeutically effective amount in combination.

According to yet another aspect of the present invention, a method of modulating effects of Interferon-β on microglia includes administering Interferon-β, and administering one or more phosphodiesterase inhibitors in a sufficient amount such that an increase in a microglial production of an inflammatory mediator caused by the Interferon-β is reduced.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a graph showing IL-12 production by microglia in the presence of various doses of IFNβ, along with the mRNA expression of IL-12 p35 and p40;

FIGS. 2A-2D are graphs showing up-regulation of microglial production of inflammatory mediators, TNFα, IL-1β, IL-6 and NO, by IFNβ;

FIGS. 3A and 3B are panels showing mRNA expression of inflammatory mediators in LPS-stimulated and non-stimulated microglia, respectively;

FIG. 4 is a graph showing increased microglial IL-10 production by IFNβ, along with the mRNA expression of IL-10;

FIG. 5 is a set of panels showing the effects of IFNβ on the mRNA expression of APC related molecules in IFNγ-treated microglia;

FIGS. 6A-6D are graphs showing levels of cytokines released from MOG₃₅₋₅₅-specific T cells in the presence of various doses of IFNβ;

FIGS. 7A-7D are graphs showing the effects of IFNβ on production of inflammatory mediators by macrophages;

FIGS. 8A and 8B are graphs showing the suppression of IFNβ-enhanced production of NO and TNFα by the phosphodiesterase inhibitor ibudilast;

FIGS. 9A and 9B are graphs illustrating the suppression of IL-12 p70 production by microglia as measured by Enzyme-Linked Immunosorbent Assay (ELISA);

FIG. 10 is a set of panels showing suppression of IL-12 p35 and p40 mRNA expression by microglia as measured by RT-PCR; and

FIG. 11 is a graph showing cytokine production by MOG₃₅₋₅₅-specific T cells.

### DESCRIPTION OF THE EMBODIMENTS

The embodiments will now be described with reference to the accompanying drawings, wherein like reference numerals designate corresponding or identical elements throughout the various drawings.

As discussed above, there has been a long standing need to evaluate the effects of IFNβ on microglial cells and how this interaction may be modulated. Therefore, to investigate the effects of IFNβ on MS pathology, the effects on microglial functions as either effecter cells or APC in the CNS are observed. In the experiments described below, the effects of IFNβ on production of inflammatory mediators, such as TNFα, IL-6, IL-1β, NO, IL-12 and the expression of the molecules critical for antigen presentation by microglia are examined. Effects of IFNβ on Th1 differentiation is also evaluated by using myelin oligodendrocyte glycoprotein (MOG)-specific T cells and INFγ-treated microglia as APC. Also, modulation of the effects of IFNβ on microglia by various biochemical agents is discussed.

Specifically, compositions and methods designed to assess and modulate the effects of IFNβ on the microglia are discussed below. In particular, the microglial production of various inflammatory mediators of demyleination is evaluated. Modulation of the IFNβ-induced effects are then evaluated in an attempt to promote the beneficial effects of IFNβ, while attenuating their deleterious effects.

Samples used for the experiments were prepared as described below. Microglia were isolated from the primary mixed glial cell cultures from newborn C57BL/6J mice on the 14th day, by the “shaking off” method previously described (Suzumura A, Mezitis S G E, Gonatas N, Silberberg D H. “MHC antigen expression on bulk isolated macrophage-microglia from newborn mouse brain: induction of Ia antigen expression by gamma-interferon.” J Neuroimmunol 1987;15:263-278, the contents of which are hereby incorporated by reference in their entirety); the purity of the cultures was 97% to 100% as determined by immunostaining for the Fc receptor. The cultures were maintained with Dulbecco's modified Eagle's minimum essential medium supplemented with 10% fetal calf serum, 5 μg/ml bovine insulin, and 0.2% glucose.

Peritoneal macrophages were collected from the same strain of mice that were intraperitoneally injected with thioglycolate 48 hours prior to collection. Macrophages were cultured with the same medium as microglia.

The effects of IFNβ on cytokines and NO production were evaluated in the flooding manner. Microglia and peritoneal macrophages were cultured in 24-well culture plates at a concentration of 1×10⁶/ml with or without 1 μg/ml LPS (lipopolysaccharide) for 24 hours in the presence of a graded concentration of IFNβ. The supernatant was then collected and stored at −70° C. until assessed. From the remaining cells, total RNA was extracted, following the guanidinium thiocyanate method (RNeasy Mini Kit;QIAGEN). The cDNA encoding mouse TNFα, IL-1β, IL-6, iNOS (inducible nitric oxide synthetase), and IL-12, was generated by reverse transcription-polymerase chain reaction (RT-PCR) using Superscript II (INVITROGEN), and Ampli Taq DNA polymerase (APPLIED BIOSYSTEMS) with the specific primers shown in Table 1.

TABLE 1
The sequence of primers for RT-PCR

| | | | |
|---|---|---|---|
| TNFα | Forward Primer | 5′-ATGAGCACAGAAAGCATGATCCGC | (SEQ ID NO: 1) |
| | Reverse Primer | 5′-ATGAGCACAGAAAGCATGATCCGC | (SEQ ID NO: 2) |
| IL-6 | Forward Primer | 5′-ATGAAGTTCCTCTCTGCAAGAGACT | (SEQ ID NO: 3) |
| | Reverse Primer | 5′-CACTAGGTTTGCCGAGTAGATCTC | (SEQ ID NO: 4) |
| IL-1β | Forward Primer | 5′-ATGGCAACTGTTCCTGAACTCAACT | (SEQ ID NO: 5) |
| | Reverse Primer | 5′-CAGGACAGGTATAGATTCTTTCCTTT | (SEQ ID NO: 6) |
| iNOS | Forward Primer | 5′-CCCTTCCGAAGTTTCTGGCAGCAGC | (SEQ ID NO: 7) |
| | Reverse Primer | 5′-GGCTGTCAGAGCCTCGTGGCTTTGG | (SEQ ID NO: 8) |
| IL-12p35 | Forward Primer | 5′-GACTTGAAGATGTACCAGACAG | (SEQ ID NO: 9) |
| | Reverse Primer | 5′-GAGATGAGATGTGATGGGAG | (SEQ ID NO: 10) |
| IL-12p40 | Forward Primer | 5′-GAAGTTCAACATCAAGAGCAGTAG | (SEQ ID NO: 11) |
| | Reverse Primer | 5′-AGGGAGAAGTAGGAATGGGG | (SEQ ID NO: 12) |
| class II | Forward Primer | 5′-AAGAAGGAGACTGTCTGGATGC | (SEQ ID NO: 13) |
| MHC | Reverse Primer | 5′-TGAATGATGAAGATGGTGCCC | (SEQ ID NO: 14) |
| B7-1 | Forward Primer | 5′-CCATGTCCAAGGCTCATTCT | (SEQ ID NO: 15) |
| | Reverse Primer | 5′-TTCCCAGCAATGACAGACAG | (SEQ ID NO: 16) |
| B7-2 | Forward Primer | 5′-GTAGACGTGTTCCAGAACTT | (SEQ ID NO: 17) |
| | Reverse Primer | 5′-TCTCACTGCCTTCACTCTGCAT | (SEQ ID NO: 18) |
| ICAM-1 | Forward Primer | 5′-TTCACACTGAATGCCAGCTC | (SEQ ID NO: 19) |
| | Reverse Primer | 5′-GTCTGCTGAGACCCCTCTTG | (SEQ ID NO: 20) |
| β-actin | Forward Primer | 5′-GTGGGCCGCTCTAGGCACCAA | (SEQ ID NO: 21) |
| | Reverse Primer | 5′-CTCTTTGATGTCACGCACGATTTC | (SEQ ID NO: 22) |

Cytokine production was measured with an ELISA kit specific for IL1, IL-6, TNFα, and IL-12 p70 (TECHNE Corp. MN). NO production was determined by Griess reaction. Briefly, a 50 μl aliquot of supernatants were mixed with an equal volume of Griess reagent (0.1% N-ethylenediarnine dihydrochloride, 1% sulfanilamide, and 2.5% phosphoric acid) and incubated for 15 minutes at room temperature, and the absorbance was read at 540 nm on a microtiter plate reader. Nitrite concentrations were calculated from a standard curve of NaNO₂ (Yoshikawa M, Suzumura A, Tamaru T, et al. “Effects of phosphodiesterase inhibitors on microglia.” Multiple Scler 1999;5: 126-133, the contents of which are hereby incorporated by reference in their entirety).

The effects of IFNβ on mRNA expression of class II MHC antigens and costimulatory molecules were evaluated in the following manner. Microglia at a concentration of 1×10⁶ /ml were plated on a well of a 24 well plate and stimulated with 1 ng/ml INFγ in the presence of a graded dose of IFNβ for 24 hours, then the cells were harvested to extract RNA as described hereinabove. The mRNA expression for class II MHC antigen, B7-1, B7-2, and ICAM-1 were examined by RT-PCR, with the specific primers as recited in Table 1 above.

To assess the effects on Th1 differentiation, the following techniques were employed. Myelin oligodendrocyte glycoprotein (MOG) 35-55-specific T cells were prepared from C57BL/6J mice immunized with 200 μg/head of MOG 35-55 (kindly provided by Dr. H. Offner, Oregon Health and Science University, Portland, Oreg.) in Freund's complete adjuvant containing 300 μg/head Mycobacterium tuberculosis H37RA (DIFCO Lab., Detroit, Mich.). Ten days later, lymph node cells were harvested, and 8×10⁶ cells were cultured with 20 μg/ml MOG 35-55 for 48 hours. After expanded in the presence of IL-2 for 7 days, MOG 35-55-specific T cells were used for following experiments. Microglia treated with 1 ng/ml IFNγ for 24 hr in the presence of a graded dose of IFNβ (0-10⁴ U/ml) were used as antigen presenting cells after washed 3 times with PBS. Also, 1×10⁶/ml MOG 35-55specific T cells were cultured on the above-treated microglia with or without 20 μg/ml MOG 35-55. Supernatants were collected at 48 hours and assessed for the contents of IFNγ, IL-4 and IL-10 by ELISA kits (BD BIOSCIENCES, San Diego, Calif.).

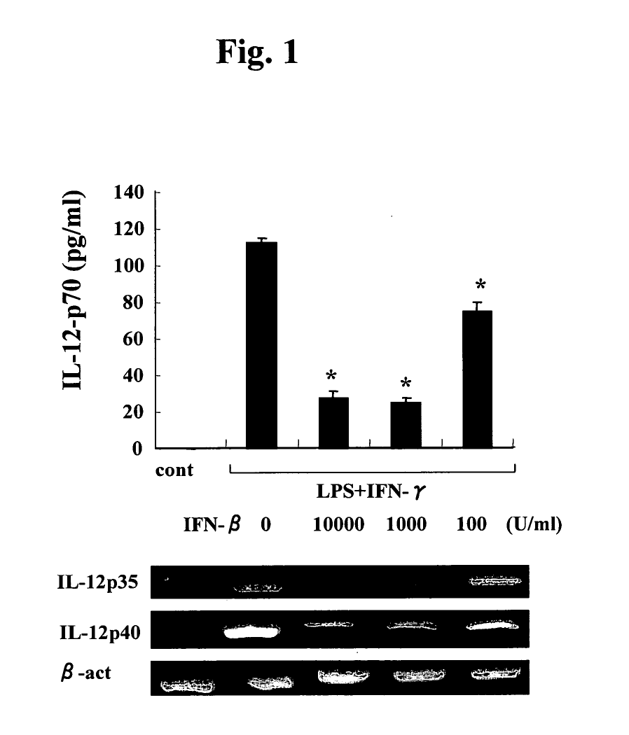

In order to evaluate candidate agents that protect against IFNβ-induced elevation of inflammatory products, the effects of cAMP-elevating agents were investigated. In particular, the phosphodiesterase inhibitor (PDEI) ibudilast was evaluated by culturing microglia at a concentration of 1×10⁶/ml in 24-well culture plates with 10⁴ U/ml IFNβ and/or 1 μg/ml LPS, in the presence of 1-100 μM ibudilast for 24 hours. Supernatants were then collected and assessed for the cytokine contents by ELISA and for NO by Griess method. Results of the experiments are discussed below by referring to FIGS. 1-11.

As previously reported, microglia produced IL-12 p70, the functional heterodimer, upon stimulation with LPS and IFNγ (Suzumura et al., 1987, supra). FIG. 1 is a graph showing the IL-12 production by microglia in the presence of a graded dose of IFNβ (0-10⁴ U/ml), and it indicates that IFNβ dose-dependently suppressed IL-12 p70 production with microglia. Since the functional heterodimer was composed of p35 and p40, the mRNA expression for those proteins was examined for the same samples. Again, IFNβ dose-dependently suppressed the expression of both p35 and p40 mRNA (FIG. 1).

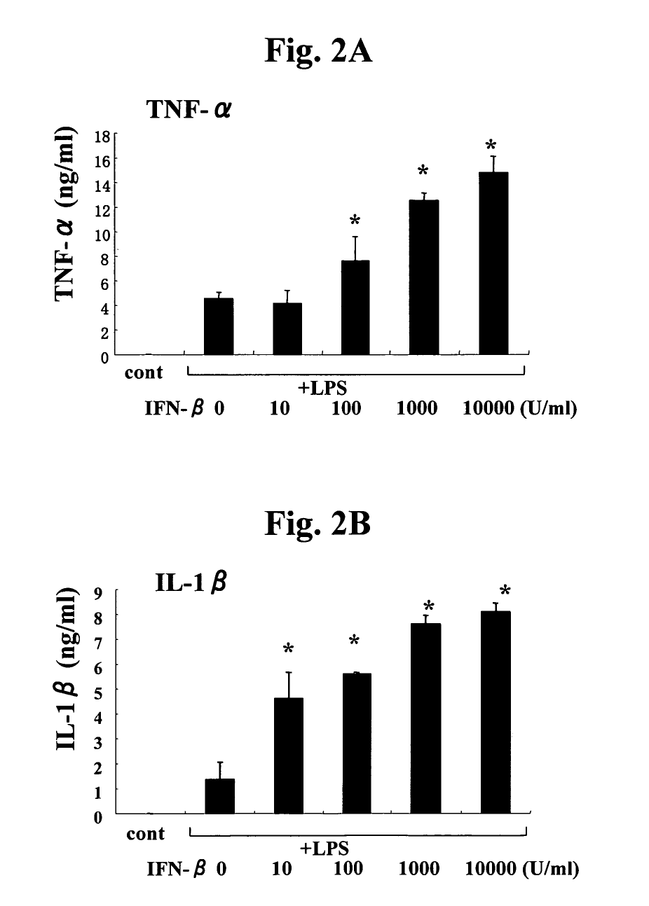

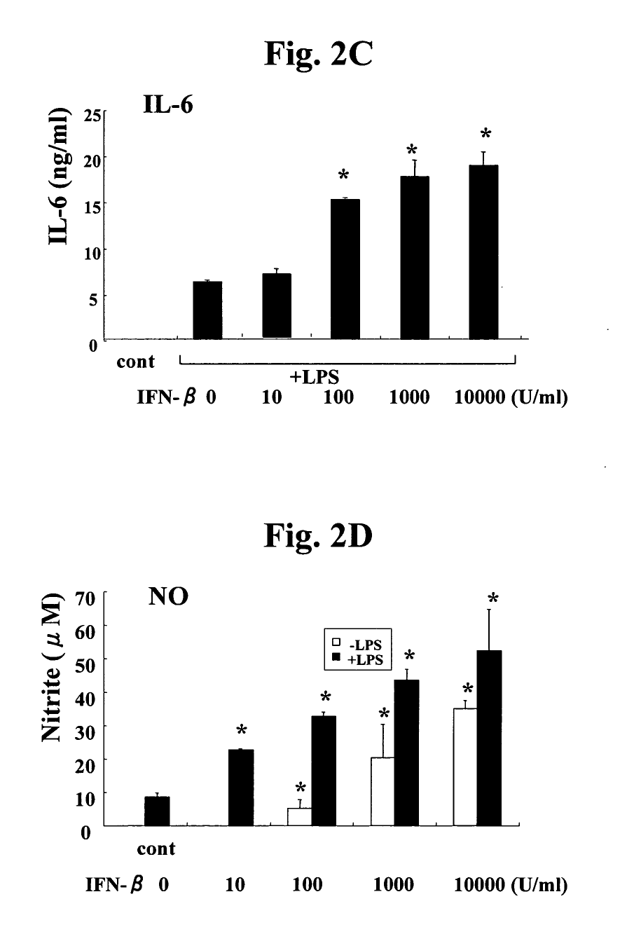

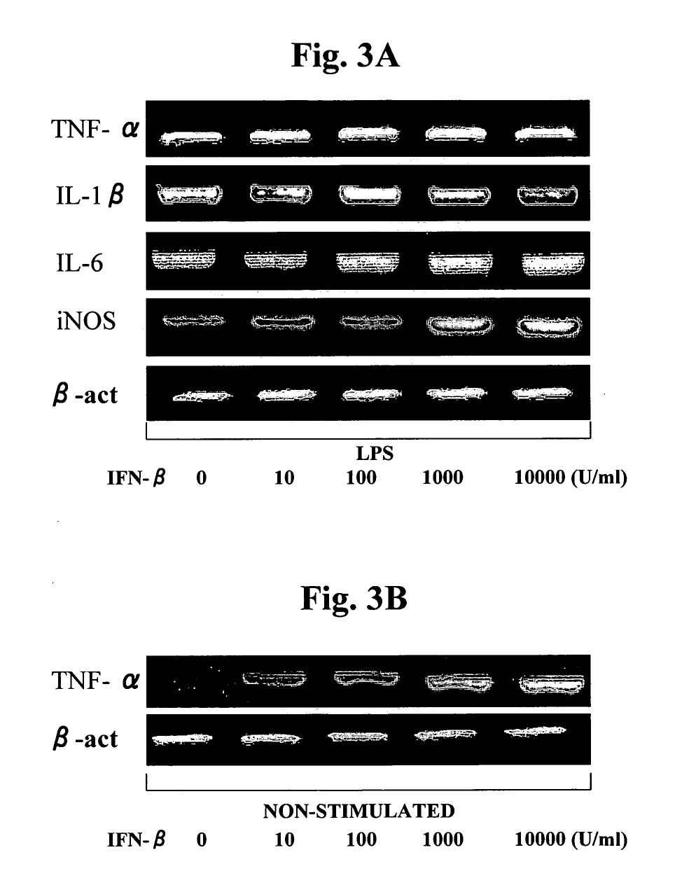

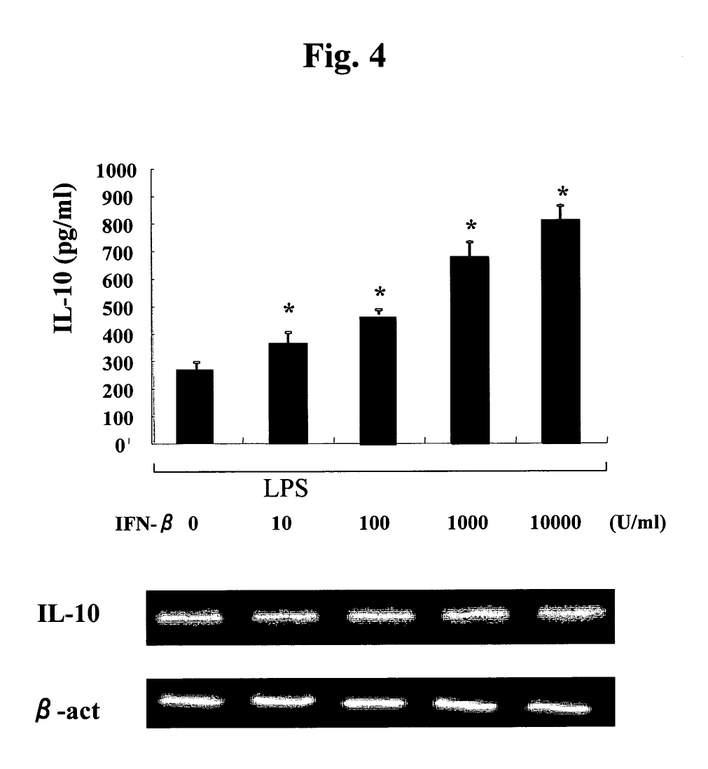

Microglia also produced a variety of inflammatory cytokines, such as TNFα, IL-1β, and IL-6 in response to LPS. As shown in the graphs of FIGS. 2A-2C, IFNβ dose-dependently increased the release of these inflammatory cytokines from microglia, as assessed by ELISA. IFNβ also increased the production of NO either in non-stimulated or LPS-stimulated microglia, as determined with Griess reagent (FIG. 2D). The mRNA expression for TNFα, IL-1β, IL-6 and iNOS in LPS-stimulated microglia were also up-regulated with IFNβ in a dose-dependent manner, though the changes in IL-1β mRNA expression were mild (FIG. 3A). IFNβ enhanced the expression of TNFα even in non-stimulated microglia (FIG. 3B). Furthermore, IFNβ dose-dependently increased the production of anti-inflammatory cytokine, IL-10 as assessed by ELISA for secreted protein and with RT-PCR for its mRNA expression (FIG. 4).

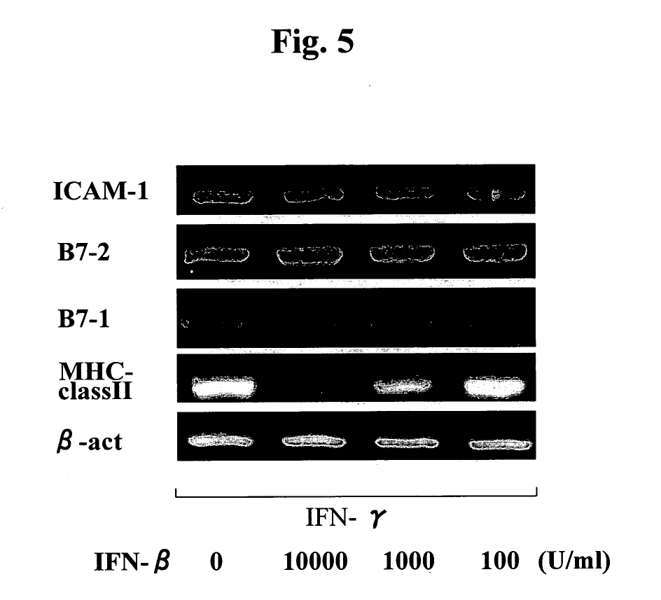

The expression of mRNA for class II MHC antigen, B7-1, B7-2, and ICAM-1 were examined using β-actin as an internal control. Although unstimulated microglia did not express class II MHC mRNA, IFNγ induced the expression. IFNβ at the dose of 10⁴ and 10³ U/ml suppressed class II MHC mRNA expression induced by IFNγ. It also slightly suppressed the expression of B7-1, but had no significant effect on the mRNA expression for B7-2 and ICAM-1 (FIG. 5).

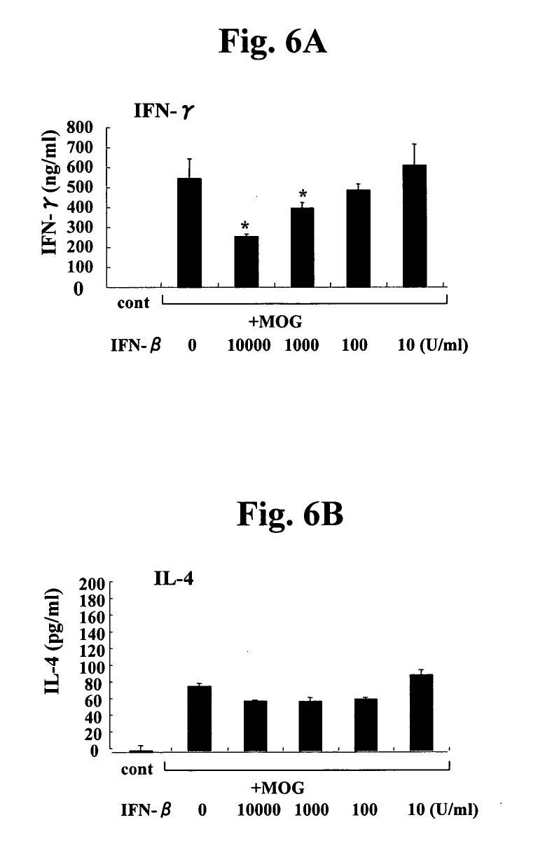

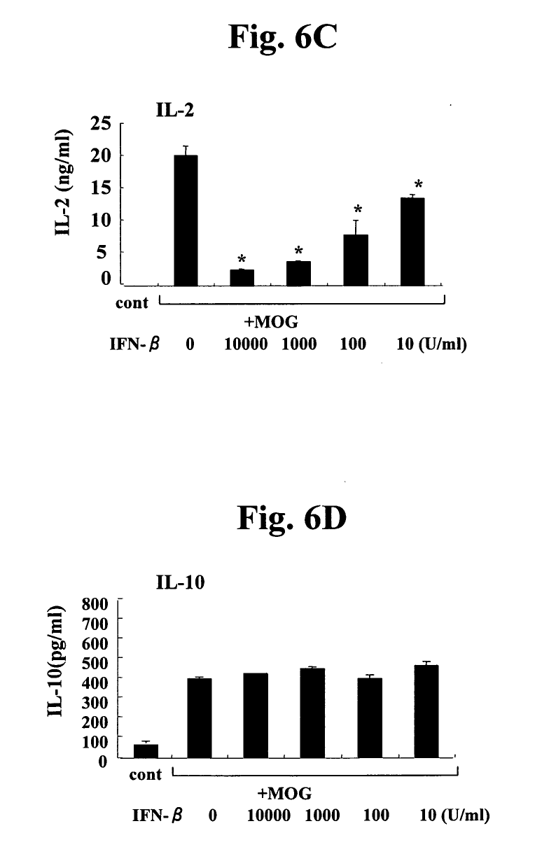

MOG-specific T cells produced high amounts of IFNγ and IL-2, when they were stimulated with MOG 35-55 in the presence of IFNγ-treated microglia as an APC, indicating that they had differentiated to a Th1 phenotype. FIGS. 6A-6D show the amounts of IFNγ, IL-2, IL-4 and IL-10 in the presence of IFNβ at different doses. When IFNβ was added with IFNγ, IFNβ significantly suppressed the differentiation to Th1 as observed in the decrease of IFNγ and IL-2 (FIGS. 6A and 6C). The production of IL-4 and IL-10 with these cells was low and the changes of the production were mild (FIGS. 6B and 6D), suggesting that the majority of the MOG-reactive T cells had developed into a Th1 phenotype in response to MOG, and that IFNβ did not significantly affect the differentiation of Th2 cells.

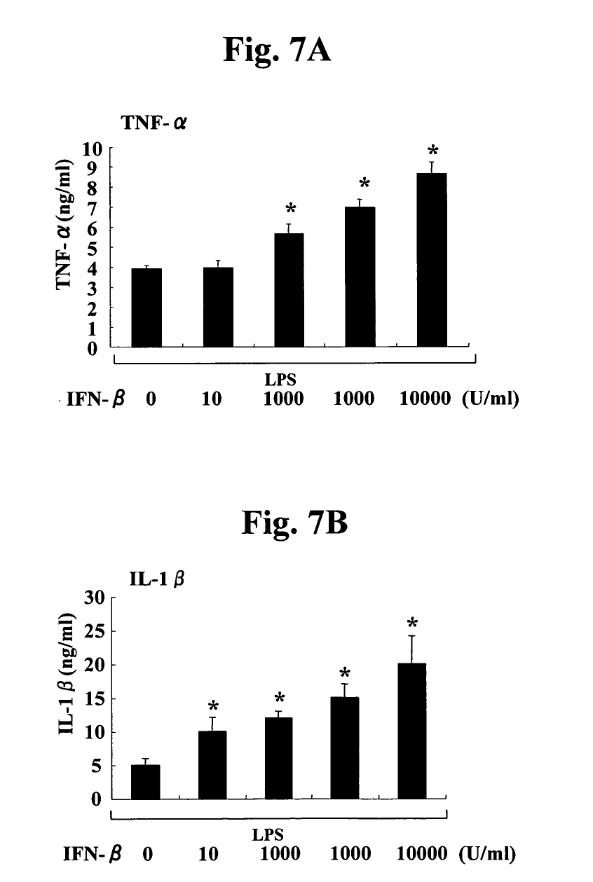

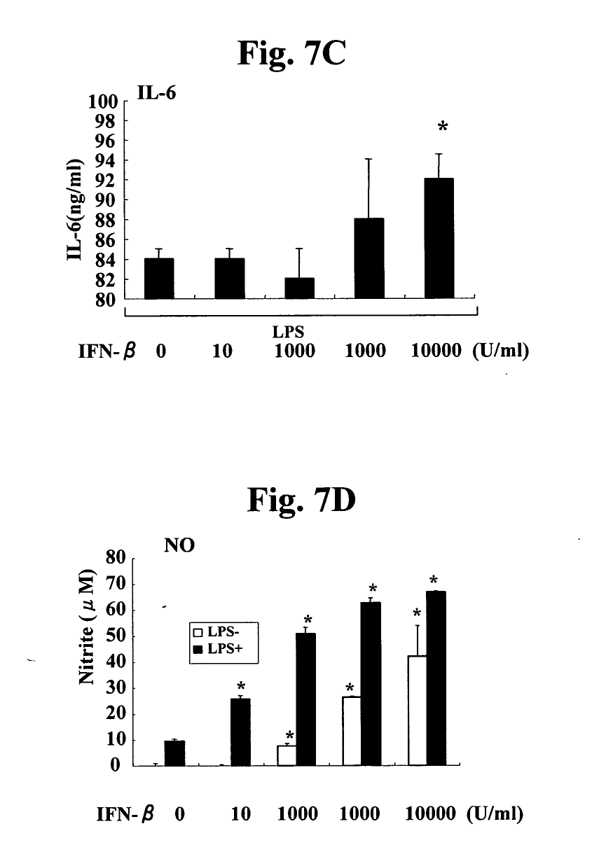

Since IFNβ enhanced the production of inflammatory cytokines with microglia, whether or not IFNβ had similar effects on macrophages was also examined. As a result, IFNβ dose-dependently increased the TNFα, IL-1β, IL-6, and NO production with LPS-stimulated macrophages (FIGS. 7A-7D). IFNβ also dose-dependently increased NO production with unstimulated macrophages (FIG. 7D).

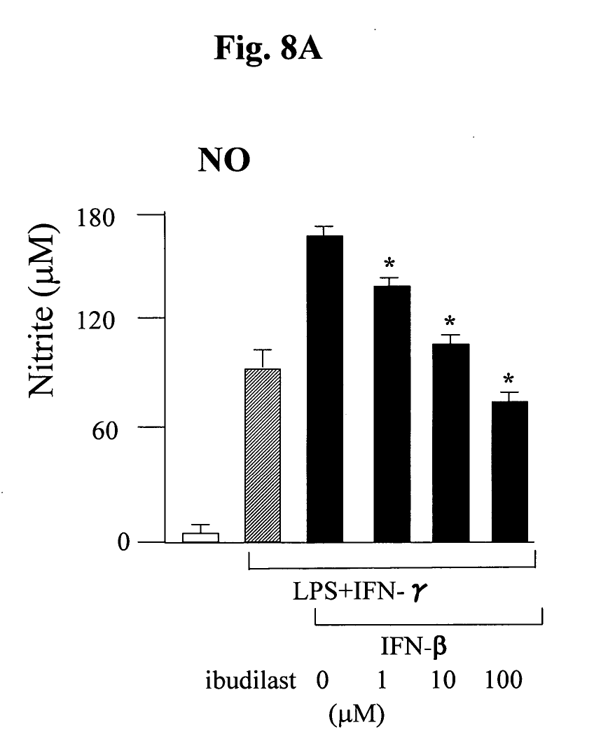

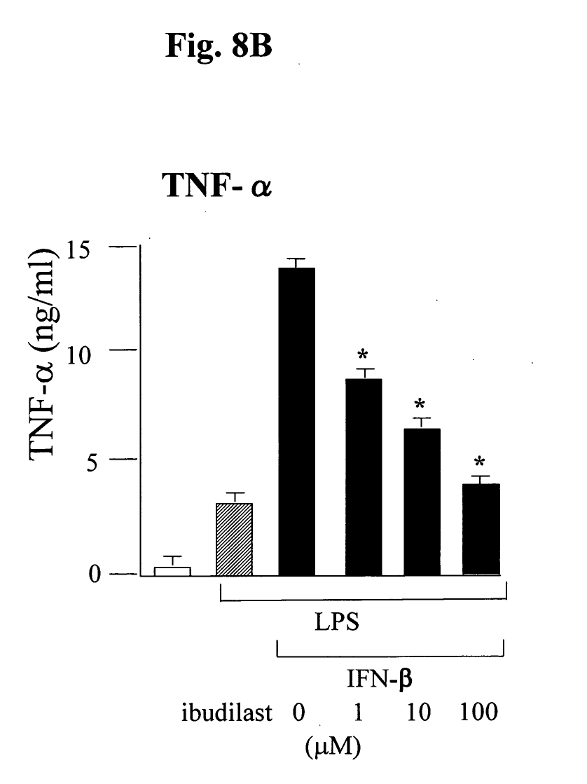

Finally, the effects of the phosphodiesterase inhibitor (PDEI), ibudilast, on IFNβ-induced increase of NO and TNFα production with microglia were examined. Ibudilast dose-dependently suppressed the NO production enhanced by IFNβ in LPS+IFNγ-stimulated microglia (FIG. 8A). Ibudilast dose-dependently suppressed the TNFα production enhanced by IFNβ in LPS-stimulated microglia (FIG. 8B). Ibudilast also suppressed NO and TNFα production with IFNβ-treated macrophages (data not shown).

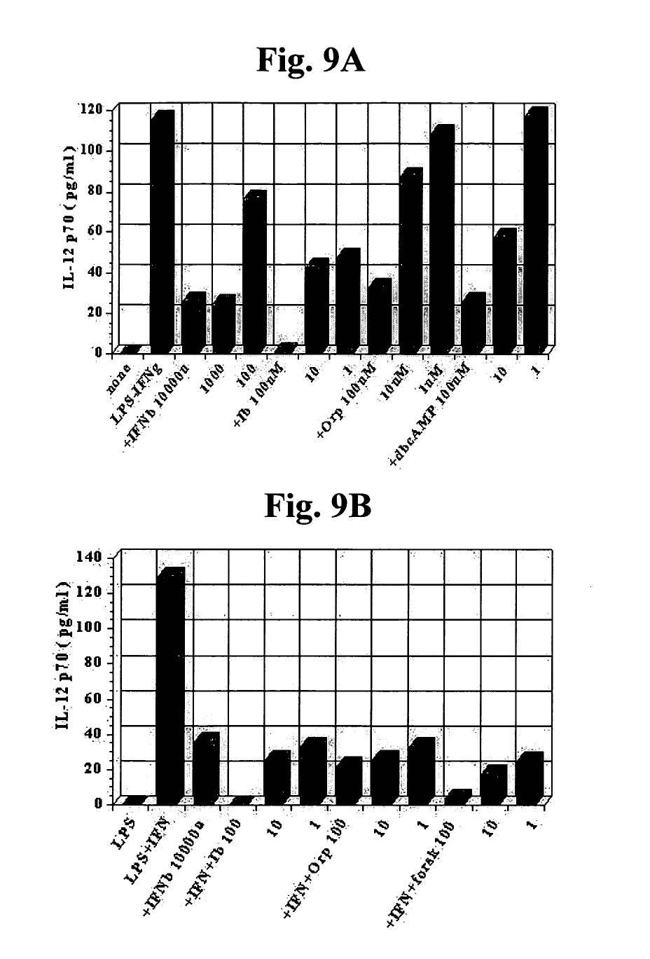

In addition, the direct effects of PDEIs on microglia were investigated in the absence of IFNβ. As shown previously, microglia produced IL-12 p70 upon response to LPS and IFNγ stimulation (Suzumura A, Mezitis S G E, Gonatas N, Silberberg D H. “MHC antigen expression on bulk isolated macrophage-microglia from newborn mouse brain: induction of Ia antigen expression by gamma-interferon.” J Neuroimmunol 1987;15:263-278). Ibudilast, orprinone, dibutyryl cAMP, and forskolin dose-dependently suppressed IL-12 production by microglia (FIG. 9A). IFNβ also suppressed IL-12 production in a dose-dependent manner (FIG. 9A). When the PDEIs were applied with IFNβ, additive or synergistic suppression of IL-12 was observed (FIG. 9B). In FIGS. 9A and 9B, the concentrations of LPS, IFNγ and IFNβ were 1 μg/ml, 100 ng/ml and 1000 U/ml, respectively, and the PDEIs were added in various doses (1-100 μM/ml). The following Tables 2 and 3 also show a suppression of IL-12 production with PDEI alone (Table 2) and with PDEI and IFNβ (Table 3), respectively.

TABLE 2
Suppression of IL-12 production with PDEI

| | | IL-12 p70 (pg/mL) |
|---|---|---|
| None | | 0 |
| LPS (1 μg/mL) + IFNγ | | 118 ± 13 |
| (1 ng/mL) | | |
| +Ibudilast | 1 μM | 53 ± 4* |
| | 10 μM | 48 ± 5* |
| | 100 μM | <7^{a}* |
| +Orprinone | 1 μM | 107 ± 20 |
| | 10 μM | 88 ± 14* |
| | 100 μM | 32 ± 11* |
| +dbcAMP | 1 μM | 122 ± 23 |
| | 10 μM | 59 ± 18* |
| | 100 μM | 23 ± 8* |

The data indicate mean ± SD (n = 9).
*P < 0.001 compared with LPS + IFNγ-stimulated microglia.
^{a}Below the detectable level (less than 7 pg/mL).
The data indicate mean ± SD (n = 9).
*P < 0.001 compared with LPS + IFNγ-stimulated microglia.
**P < 0.001 compared with LPS + IFNγ + IFNβ-stimulated microglia.
^{a}Below the detectable level (less than 7 pg/mL).

TABLE 3
Synergistic suppression of IL-12 production with IFNβ and PDEI

| | | | IL-12 p70 (pg/mL) |
|---|---|---|---|
| LPS (1 μg/mL) + IFNγ (1 ng/mL) | | | 118 ± 13 |
| +IFNβ | 10² | U/mL | 78 ± 18* |
| | 10³ | U/mL | 21 ± 12* |
| | 10⁴ | U/mL | 23 ± 4* |
| +IFNβ 10² U/mL + ibudilast | 1 | μM | 48 ± 11** |
| | 10 | μM | 43 ± 6** |
| | 100 | μM | <7^{a}** |
| +IFNβ 10² U/m + orprinone | 1 | μM | 67 ± 14 |
| | 10 | μM | 44 ± 7** |
| | 100 | μM | 40 ± 9** |
| +IFNβ 10² U/m + forskoline | 1 | μM | 47 ± 10** |
| | 10 | μM | 34 ± 6** |
| | 100 | μM | <7^{a}** |

The data indicate mean ± SD (n = 9).
*P < 0.001 compared with LPS + IFNγ-stimulated microglia.
**P < 0.001 compared with LPS + IFNγ + IFNβ-stimulated microglia.
^{a}Below the detectable level (less than 7 pg/mL).

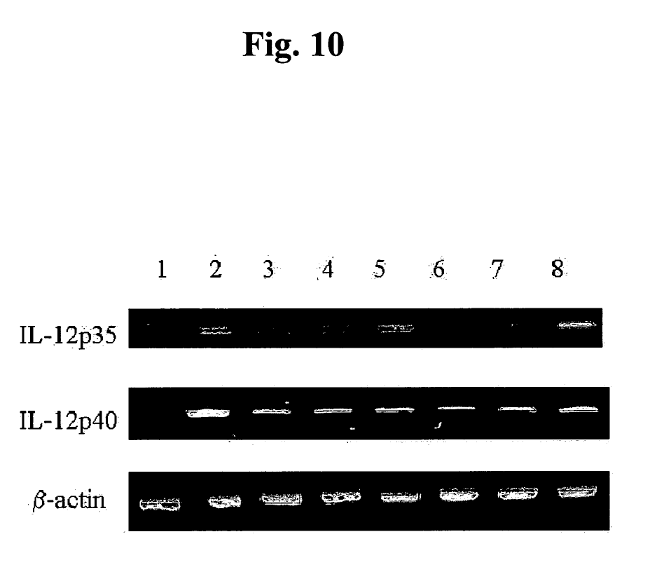

Since functional IL-12 is a heterodimer of IL-12 p35 and p40, we examined the expression of p35 and p40 mRNA in the above treated microglia. Again, IFNβ and ibudilast dose-dependently suppressed p35 and p40 mRNA expression in LPS and IFNγ-treated microglia (FIG. 10).

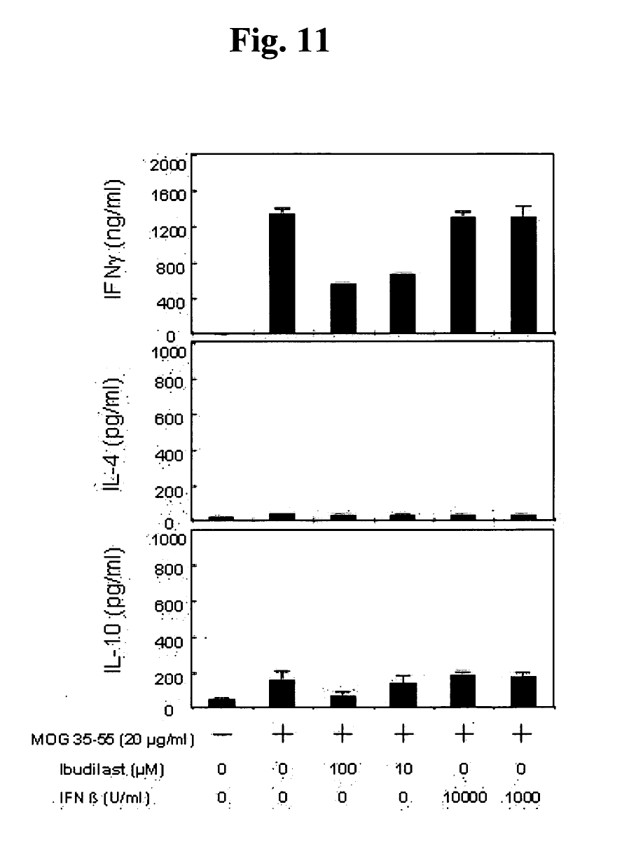

After adding MOG 35-55, MOG-specific T cells produced high amounts of IFNγ, but very little IL-4 or IL-10, indicating that these cells were sensitized with MOG 35-55 and that they had already differentiated into Th1 cells in the presence of antigen presented by microglia. Ibudilast significantly suppressed the production of IFNγ with these cells, suggesting that it interfered with the development of Th1, most likely by suppressing the production of IL-12. In contrast, the effects of IFNβ on cytokine production by MOG-specific T cells was not remarkable (FIG. 11).

Further experiments demonstrated that PDEIs as well as other cAMP-elevating agents suppressed IL-12 production by microglia. T cells specific for MOG 35-55 differentiated into a Th1 phenotype when MOG 35-55 was presented by microglia. PDEI also blocked this Th1 differentiation. IFNβ similarly suppressed IL-12 production by microglia as reported (Satoh J, Lee Y B, Kim S U. “T-cell costimulatory molecules B71 (CD80) and B7-2 (CD86) are expressed in human microglia but not in astrocytes in culture.” Brain Res 1995; 704: 92-96), but did not directly block the differentiation of T cells into Th1. Although IFNβ reportedly induces immune deviation in patients with MS (R. A. Rudick, R. M. Ransohoff, J. C. Lee, R. Peppler, M. Yu, P. M. Mathisen, V K. Touhy, “In vivo effects of interferon beta-1a on immunosuppressive cytokines in multiple sclerosis.” Neurology 50 (1998) 1294-1300), it may be less effective than PDEI when antigens are presented by microglia in the CNS. As has been shown previously, microglia usually very weakly express class II MHC antigen, but was induced to express the antigen by IFNγ (Ossege L M, Sindern E, Voss B, Malin J P “Immunomodulatory effects of IFNβ-1b on the mRNA-expression of TGFβ-1 and TNFα in vitro.” Immunopharmacol 1999;43:39-46). It has been shown that IFNγ also induced co-stimulatory molecules such as B7-1 and B7-2 (Menendez Iglesias B, Cerase J, Ceracchini C, Levi G, Aloisi F. “Analysis of B7-1 and B7-2 costimulatory ligands in cultured mouse microglia: upregulation by interferon-gamma and lipopolysaccharide and downregulation by interleukin-10, prostaglandin E2 and cyclic AMP-elevating agents.” J Neuroimmunol 1997;72: 83-93), making microglia sufficient for professional antigen presenting cells. It is suggested that antigen presentation in the CNS is critical in the development of autoimmune demyelination (Yoshikawa M, Suzumura A, Tamaru T, et al. “Effects of phosphodiesterase inhibitors on microglia.” Multiple Scler 1999;5: 126-133). T cells sensitized to some CNS antigens can expand to induce pathology when they encounter antigen and antigen-presenting cells in the CNS. Therefore, it is a very notable finding that ibudilast suppressed differentiation of MOG 35-55-specific T cells into a Th1 phenotype with MOG 35-55 presented by microglia.

The precise mechanism of how PDEIs suppress IL-12 production by microglia remains to be clarified. However, since suppression of activation and/or translocation of NF-κB results in suppression of inflammatory cytokines including IL-12, it is likely that PDEI suppresses IL-12 production through NK-κB deactivation (Suzumura et al., 1987 supra). It has also been shown that IL-12 signaling through a Janus kinase (JAK)-STAT pathway is critical in the induction of Th1 differentiation and that the blocking of this signal pathway results in prevention of Th1 differentiation and inflammatory demyelination in EAE (Suzumura A, Sawada M, Takayanagi T. “Production of interleukin-12 and the expression of its receptors by murine microglia.” Brain Res 1998;787:139-142). Since ibudilast did not only suppress IL-12 production by microglia, but also suppressed Th1 differentiation of MOG-specific T cells, it is possible that PDEI may also affect the JAK-STAT pathway in IL-12 signaling.

As shown in Table 3, PDEIs and IFNβ functioned additively or synergistically to suppress IL-12 production with microglia. In the clinical trial, Type III PDEI pentoxifylline was shown to synergistically function with IFNβ to reduce production of inflammatory cytokines, and up-regulate an anti-inflammatory cytokine IL-10 in peripheral blood mononuclear cells from patients with active MS (Hickey W F, Kimura H. “Perivascular microglial cells of the CNS are bone marrow-derived and present antigen in vivo.” Science 1988;239(4837):290-292). PDEIs are widely and safely used for the treatment of stroke, asthma, or heart failure in Japan.

In a method of treating MS according to one embodiment of the present invention, IFNβ and PDEIs are utilized in combination. As seen from FIGS. 2A-2D, when IFNβ is used alone, the production of inflammatory mediator is increased. However, as FIGS. 8A and 8B indicate, by administering IFNβ and PDEI in combination as in the method according to one embodiment of the present invention, such an increase is compensated and the level of the inflammatory mediator is further lowered by the addition of PDEI. The release of inflammatory signaling molecules by IFNβ is thought to give rise to the negative symptoms associated with its clinical use. However, the suppression of this release by PDEIs may result in improvement both in the negative side effects of IFNβ, as well as an improvement in its therapeutic efficacy. IFNβ and PDEIs may be administered in the same solution or in separate solutions that are applied simultaneously or in close temporal proximity. Such temporal regimens suitable for the administration of IFNβ and PDEIs may be deduced.

As used herein, the terms “therapeutic” and/or “effective” amounts mean an agent utilized in an amount sufficient to treat, combat, ameliorate, prevent or improve a condition or disease of a patient. These disease conditions include MS.

Phosphodiesterase inhibitors and IFNβ may be administered orally, for example, with an inert diluent, typically an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, waters, chewing gums, and the like. The amount of the compounds consisting of embodiments of the present invention will be such that a suitable dosage will be provided in the administered amount.

Tablets, pills, capsules, troches and the like may contain the following ingredients: a binder, such as micro-crystalline cellulose, gum tragacanth or gelatin; an excipient, such as starch or lactose; a disintegrating agent, such as alginic acid, Primogel, corn starch and the like; a lubricant, such as magnesium stearate or Sterotes; a glidant, such as colloidal silicon dioxide; a sweetening agent, such as sucrose, saccharin or aspartame; or flavoring agent, such as peppermint, methyl salicylate or orange flavoring. When the dosage unit form is a capsule it may contain, in addition to compounds comprising embodiments of the present invention, a liquid carrier, such as a fatty oil. Other dosage unit forms may contain other materials that modify the physical form of the dosage unit, for example, as coatings. Thus, tablets or pills may be coated with sugar, shellac or other enteric coating agents. A syrup may contain, in addition to the active compounds, sucrose as a sweetening agent and preservatives, dyes, colorings and flavors. Materials used in preparing these compositions should be pharmaceutically pure and non-toxic in the amounts used.

For purposes of parenteral therapeutic administration, the phosphodiesterase inhibitors and IFNβ may be incorporated into a solution or suspension. The amount of active compound in such compositions is such that a suitable dosage will be obtained.

Solutions or suspensions of phosphodiesterase inhibitors and IFNβ may also include the following components: a sterile diluent, such as water for injection, saline solution, fixed oils, polyethylene glycols, glycerine, propylene glycol or other synthetic solvents: antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity or osmolarity, such as sodium chloride or dextrose. The parenteral preparation may be enclosed in ampoules, disposable syringes or multiple dose vials made of glass or plastic.

It is to be understood that phosphodiesterase inhibitors and IFNβ may be administered in the form of a pharmaceutically acceptable salt. Examples of such salts include acid addition salts. Preferred pharmaceutically acceptable addition salts include salts of mineral acids, for example, salts of hydrochloric acid, sulfuric acid, nitric acid and the like; salts of monobasic carboxylic acids, such as, for example, acetic acid, propionic acid and the like; salts of dibasic carboxylic acids, such as maleic acid, fumaric acid, oxalic acid and the like; and salts of tribasic carboxylic acids, such as, carboxysuccinic acid, citric acid and the like.

Additionally, phosphodiesterase inhibitors may be administered in combination with other therapeutic compositions (e.g., IFNβ) in order to achieve the desired, improved conditions in the subject in need thereof.

It is to be understood, however, that for any particular subject, specific dosage regimens should be adjusted to the individual need and the professional judgement of the person administering or supervising the administration of the compound. The term “subject” as used herein means humans, and in particular humans suffering from MS.

The exact dosages of phosphodiesterase inhibitor and IFNβ to be administered will, of course, depend on the size and condition of the patient being treated and the identity of the particular phosphodiesterase inhibitor being administered.

In summary, according to one embodiment of the present invention, compositions that include interferonβ (IFNβ) and phosphodiesterase inhibitors are provided, and such compositions are useful in the treatment of MS. IFNβ has been shown to reduce exacerbation of relapsing remitting form of MS, though the exact mechanism remains to be elucidated. The effects of IFNβ on microglial functions, as either antigen presenting cells or effector cells for inflammatory demyelination, were investigated. IFNβ significantly suppressed the expression of class II MHC antigen and co-stimulatory molecules in microglia. It also suppressed microglial IL-12 production and differentiation of myelin oligodendrocyte glycoprotein (MOG)-sensitized T cells into T helper 1 phenotype, using microglia as antigen presenting cells. However, IFNβ significantly and dose-dependently enhanced production of inflammatory mediators for demyelination, such as TNFα, IL-1β, IL-6 and nitric oxide (NO). The up-regulation of inflammatory mediators was effectively suppressed with phosphodiesterase inhibitor. Side effects of IFNβ treatment may be due to elevation of pro-inflammatory cytokines, which can be reduced by co-treatment with phosphodiesterase inhibitors. Also, in a method of modulating effects of IFNβ on microglia according to one embodiment of the present invention, IFNβ is administered and produces an increase in a microglial production of an inflammatory mediator, and also at least one PDEI administered and the production of the microglial production is reduced.

Obviously, numerous modifications and variations of the present invention are possible in light of the above teachings. It is therefore to be understood that within the scope of the appended claims, the invention may be practiced otherwise than as specifically described herein.

What is claimed as new and desired to be secured by Letters Patent of the United States is:

1. A method of treating multiple sclerosis, comprising administering Interferon-β and at least one phosphodiesterase inhibitor in combination in a therapeutically effective amount.

2. The method of claim 1, wherein the administering comprises simultaneously administering the Interferon-β and the at least one phosphodiesterase inhibitor.

3. The method of claim 1, wherein the administering comprises administering the Interferon-β and the at least one phosphodiesterase inhibitor in close temporal proximity.

4. The method of claim 1, wherein the at least one phosphodiesterase inhibitor comprises ibudilast.

5. The method of claim 1, wherein the at least one phosphodiesterase inhibitor comprises orprinone.

6. The method of claim 1, wherein the at least one phosphodiesterase inhibitor comprises dibutyryl cAMP.

7. The method of claim 1, wherein the at least one phosphodiesterase inhibitor comprises forskolin.

8. The method of claim 1, wherein the administering comprises preparing at least one of the Interferon-β and the at least one phosphodiesterase inhibitor in a form of at least one pharmaceutically acceptable salt.

9. The method of claim 8, wherein the at least one pharmaceutically acceptable salt comprises at least one of an acid addition salt and a basic carboxylic salt.

10. The method of claim 9, wherein the acid addition salt comprises at least one salt selected from the group consisting of a mineral acid salt, a hydrochloric acid salt, a sulfuric acid salt and a nitric acid salt.

11. The method of claim 9, wherein the basic carboxylic salt comprises at least one salt selected from the group consisting of an acetic acid salt, a propionic acid salt, a maleic acid salt, a fumaric acid salt, an oxalic acid salt, a carboxysuccinic acid salt and a citric acid salt.

12. The method of claim 1, wherein the administering comprises preparing at least one of the Interferon-β and the at least one phosphodiesterase inhibitor in a form of solution or suspension.

13. The method of claim 12, wherein the solution or suspension further comprises at least one of a sterile diluent, an antibacterial agent, an antioxidant, a chelating agent, a buffer and a tonicity adjusting agent.

14. The method of claim 1, wherein the administering comprises preparing at least one of the Interferon-β and the at least one phosphodiesterase inhibitor in a form of tablet or capsule for oral administration.

15. The method of claim 1, wherein the Interferon-β and the at least one phosphodiesterase inhibitor are administered to a subject in need of treating multiple sclerosis.

16. A composition for treating multiple sclerosis, comprising:
Interferon-β; and
at least one phosphodiesterase inhibitor,
wherein the Interferon-β and the at least one phosphodiesterase inhibitor are included in a therapeutically effective amount in combination.

17. The composition of claim 16, wherein the at least one phosphodiesterase inhibitor comprises ibudilast.

18. The composition of claim 16, wherein the at least one phosphodiesterase inhibitor comprises orprinone.

19. The composition of claim 16, wherein the at least one phosphodiesterase inhibitor comprises dibutyryl cAMP.

20. The composition of claim 16, wherein the at least one phosphodiesterase inhibitor comprises forskolin.

21. The composition of claim 16, wherein at least one of the Interferon-β and the at least one phosphodiesterase inhibitor is in a form of at least one pharmaceutically acceptable salt.

22. The composition of claim 21, wherein the at least one pharmaceutically acceptable salt comprises at least one of an acid addition salt and a basic carboxylic salt.

23. The composition of claim 22, wherein the acid addition salt comprises at least one salt selected from the group consisting of a mineral acid salt, a hydrochloric acid salt, a sulfuric acid salt and a nitric acid salt.

24. The composition of claim 22, wherein the basic carboxylic salt comprises at least one salt selected from the group consisting of an acetic acid salt, a propionic acid salt, a maleic acid salt, a fumaric acid salt, an oxalic acid salt, a carboxysuccinic acid salt and a citric acid salt.

25. The composition of claim 16, wherein at least one of the Interferon-β and the at least one phosphodiesterase inhibitor is in a form of solution or suspension.

26. The composition of claim 25, wherein the solution or suspension further comprises at least one of a sterile diluent, an antibacterial agent, an antioxidant, a chelating agent, a buffer and a tonicity adjusting agent.

27. The composition of claim 16, wherein at least one of the Interferon-β and the at least one of phosphodiesterase inhibitor is in a form of tablet or capsule for oral administration.

28. A method of modulating effects of Interferon-β on microglia, comprising:
administering Interferon-β; and
administering at least one phosphodiesterase inhibitor in a sufficient amount such that an increase in a microglial production of an inflammatory mediator caused by the Interferon-β is reduced.

29. The method of claim 28, wherein the at least one phosphodiesterase inhibitor comprises ibudilast.

30. The method of claim 28, wherein the at least one phosphodiesterase inhibitor comprises orprinone.

31. The method of claim 28, wherein the at least one phosphodiesterase inhibitor comprises dibutyryl cAMP.

32. The method of claim 28, wherein the at least one phosphodiesterase inhibitor comprises forskolin.

33. The method of claim 28, wherein the administering at least one phosphodiesterase inhibitor comprises preparing the at least one phosphodiesterase inhibitor in a form of one of an acid addition salt and a basic carboxylic salt.

34. The method of claim 33, wherein the acid addition salt comprises at least one salt selected from the group consisting of a mineral acid salt, a hydrochloric acid salt, a sulfuric acid salt and a nitric acid salt.

35. The method of claim 33, wherein the basic carboxylic salt comprises at least one salt selected from the group consisting of an acetic acid salt, a propionic acid salt, a maleic acid salt, a fumaric acid salt, an oxalic acid salt, a carboxysuccinic acid salt and a citric acid salt.

36. The method of claim 28, wherein the administering at least one phosphodiesterase inhibitor comprises preparing the at least one phosphodiesterase inhibitor in a form of solution or suspension.

37. The method of claim 28, wherein the administering Interferon-β comprises preparing the Interferon-β in a form of one of an acid addition salt and a basic carboxylic salt.

38. The method of claim 37, wherein the acid addition salt comprises at least one salt selected from the group consisting of a mineral acid salt, a hydrochloric acid salt, a sulfuric acid salt and a nitric acid salt.

39. The method of claim 37, wherein the basic carboxylic salt comprises at least one salt selected from the group consisting of an acetic acid salt, a propionic acid salt, a maleic acid salt, a fumaric acid salt, an oxalic acid salt, a carboxysuccinic acid salt and a citric acid salt.

40. The method of claim 28, wherein the administering Interferon-β comprises preparing the Interferon-β in a form of solution or suspension.

41. The method of claim 28, wherein the inflammatory mediator is nitric oxide.

42. The method of claim 28, wherein the inflammatory mediator is TNFα.

43. The method of claim 28, wherein the microglial production is stimulated by lipopolysaccharide.

44. The method of claim 28, wherein the Interferon-β is administered in an amount of 10000 U/ml and the sufficient amount of the at least one phosphodiesterase inhibitor is 100 μM/ml.

45. The method of claim 28, wherein the Interferon-β is administered in an amount of 100 U/ml and the sufficient amount of the at least one phosphodiesterase inhibitor is 100 μM/ml.

46. The method of claim 28, wherein the Interferon-β and the at least one phosphodiesterase inhibitor are administered to a subject in need of modulating the effects of Interferon-β on microglia.

47. The method of claim 28, wherein the Interferon-β and the at least one phosphodiesterase inhibitor are administered to a subject in need of treating multiple sclerosis.


SEQUENCE LISTING (22 sequences)

SEQ ID NO: 1 - DNA, 24 bp, Artificial Sequence
  Synthetic DNA
atgagcacagaaagcatgatccgc

SEQ ID NO: 2 - DNA, 24 bp, Artificial Sequence
  Synthetic DNA
atgagcacagaaagcatgatccgc

SEQ ID NO: 3 - DNA, 25 bp, Artificial Sequence
  Synthetic DNA
atgaagttcctctctgcaagagact

SEQ ID NO: 4 - DNA, 24 bp, Artificial Sequence
  Synthetic DNA
cactaggtttgccgagtagatctc

SEQ ID NO: 5 - DNA, 25 bp, Artificial Sequence
  Synthetic DNA
atggcaactgttcctgaactcaact

SEQ ID NO: 6 - DNA, 26 bp, Artificial Sequence
  Synthetic DNA
caggacaggtatagattctttccttt

SEQ ID NO: 7 - DNA, 25 bp, Artificial Sequence
  Synthetic DNA
cccttccgaagtttctggcagcagc

SEQ ID NO: 8 - DNA, 25 bp, Artificial Sequence
  Synthetic DNA
ggctgtcagagcctcgtggctttgg

SEQ ID NO: 9 - DNA, 22 bp, Artificial Sequence
  Synthetic DNA
gacttgaagatgtaccagacag

SEQ ID NO: 10 - DNA, 20 bp, Artificial Sequence
  Synthetic DNA
gagatgagatgtgatgggag

SEQ ID NO: 11 - DNA, 24 bp, Artificial Sequence
  Synthetic DNA
gaagttcaacatcaagagcagtag

SEQ ID NO: 12 - DNA, 20 bp, Artificial Sequence
  Synthetic DNA
agggagaagtaggaatgggg

SEQ ID NO: 13 - DNA, 22 bp, Artificial Sequence
  Synthetic DNA
aagaaggagactgtctggatgc

SEQ ID NO: 14 - DNA, 21 bp, Artificial Sequence
  Synthetic DNA
tgaatgatgaagatggtgccc

SEQ ID NO: 15 - DNA, 20 bp, Artificial Sequence
  Synthetic DNA
ccatgtccaaggctcattct

SEQ ID NO: 16 - DNA, 20 bp, Artificial Sequence
  Synthetic DNA
ttcccagcaatgacagacag

SEQ ID NO: 17 - DNA, 20 bp, Artificial Sequence
  Synthetic DNA
gtagacgtgttccagaactt

SEQ ID NO: 18 - DNA, 22 bp, Artificial Sequence
  Synthetic DNA
tctcactgccttcactctgcat

SEQ ID NO: 19 - DNA, 20 bp, Artificial Sequence
  Synthetic DNA
ttcacactgaatgccagctc

SEQ ID NO: 20 - DNA, 20 bp, Artificial Sequence
  Synthetic DNA
gtctgctgagacccctcttg

SEQ ID NO: 21 - DNA, 21 bp, Artificial Sequence
  Synthetic DNA
gtgggccgctctaggcaccaa

SEQ ID NO: 22 - DNA, 24 bp, Artificial Sequence
  Synthetic DNA
ctctttgatgtcacgcacgatttc